(19) United States
(12) Patent Application Publication
(10) Pub. No.: US 2020/0321082 A1
(43) Pub. Date: Oct. 8, 2020

(54) CONFIDENCE EVALUATION TO MEASURE TRUST IN BEHAVIORAL HEALTH SURVEY RESULTS

(71) Applicant: Ellipsis Health, Inc., San Francisco, CA (US)

(72) Inventors: Elizabeth E. Shriberg, Berkeley, CA (US); Yang Lu, Waterloo (CA); Amir Harati, Toronto (CA)

(21) Appl. No.: 16/377,090

(22) Filed: Apr. 5, 2019

(51) Int. Cl. G16H 10/20; G06N 7/00

(52) U.S. Cl. CPC G16H 10/20; G06N 7/005

(57) ABSTRACT

A behavioral health survey confidence annotation machine determines a degree of confidence in the reliability of a survey taker's responses given in a behavioral health survey. The degree of confidence reflects consistencies in the survey results themselves and data about the survey taker. The degree of confidence can also reflect consistency between results of multiple instances of the survey taken contemporaneously, i.e., within a single session with the survey taker. Culling of health survey results produces a corpus of health survey result data more greater confidence in the reliability of its results. Survey takers whose health survey results are consistently unreliable can be identified.

### FIELD OF THE INVENTION

The present invention relates generally to health survey analysis systems, and, more particularly, to a computer-implemented health survey analysis tool with significantly improved accuracy and efficacy.

### BACKGROUND

Behavioral health is and will always be a serious problem. However, the most widely used and relied-upon tools for screening for behavioral health problems rely on accurate and reliable self-reporting by the screened public. The current “gold standard” for questionnaire-based screening for depression is the PHQ-9 (Survey taker Health Questionnaire 9), a written depression health survey with nine (9) multiple-choice questions. Other similar health surveys include the PHQ-2 and the Generalized Anxiety Disorder 7 (GAD-7).

An important problem when using the PHQ-9 (or similar survey analysis tool) is a lack of veracity or other inaccuracy in the multiple choice answers marked by the user. We will often refer to veracity and accuracy in general simply as “reliability” herein. Survey taker responses can be unreliable for a number of reasons, e.g., boredom, inattention, or distraction of the survey taker while taking the test; a survey taker cheating by answering questions in a way believed to yield a particular result; or lack of ability to answer the questions due to lack of language proficiency, illiteracy, and simply not understanding the question(s).

Such unreliable responses can lead to misdiagnoses of survey takers. However, consequences of unreliable responses can extend far beyond the correctness of a diagnosis of a given survey taker. Unreliable responses can render any statistical analysis or modeling of the corpus less accurate and less useful. Examples include analysis for population assessments, for monitoring, or for assessment of therapeutic treatments including medications. Examples also include AI systems that are trained to predict depression and that use the survey data as ground truth estimates for model training and evaluation. Some percentage of the survey data used for analysis, interpretation or machine learning based models will contain problems of the types just mentioned, resulting in suboptimal interpretations and suboptimal models.

What is needed is a way to identify which survey results may be affected by lack of reliability for the reasons above, so that end users of the surveys can decide whether or not to include the surveys for their purposes. Instead of a simply binary yes/no guess at which surveys are not to be trusted, what is needed is a score, or “confidence” to represent the estimated veracity or reliability of the particular survey data. End users can then threshold the scores based on the tolerance for corruption risk in their survey data, for their particular application. In survey taker responses in health survey analysis tools.

### SUMMARY

In accordance with the present invention, a behavioral health survey confidence annotation machine determines a degree of confidence in the reliability of a survey taker's responses given in a behavioral health survey. The proposed health survey confidence annotation machine processes behavioral health survey results and outputs a score that is monotonically related to the estimated veracity of the results. The degree of confidence represented by the score reflects testing for multiple types of consistencies. These include but are not limited to consistencies of the survey answer patterns with respect to a set of prior survey data, and conditional consistencies based on characteristics of the survey taker and the survey context. The behavioral health survey confidence annotation machine can also implement a process in which the survey taker takes the same survey more than once and the behavioral health survey confidence annotation machine then computes additional reliability measures using consistencies in results of corresponding questions across the multiple surveys. In addition, the behavioral health survey confidence annotation machine can output real-time estimates that can be used to intervene in the survey administration process, resulting in potential better quality and/or cost savings for both the survey taker and the survey administration team.

Given these confidence annotations, any analysis of behavioral health survey results, e.g., statistical analysis and computational modeling through artificial intelligence (AI) such as deep machine learning, can be significantly more accurate and useful. For example, in such analysis, survey results with lower confidence can be weighted less or disregarded altogether while survey results with higher confidence can be weighted more heavily.

Identifying health survey results with relatively low confidence in the reliability thereof provides a number of significant advantages. An important one is the culling of health survey results such that a corpus of health survey result data can include only adequately reliable results. Such significantly improves the results of any analysis of the corpus as a whole, including statistical analysis and artificial intelligence (AI) analysis. Any modeling of such a corpus of health survey results can yield much better analysis.

Another significant advantage is that survey takers whose health survey results are consistently unreliable can be identified. The health survey results of these survey takers can be the result of inattentiveness, intent to influence the survey results and indications, illiteracy, or insufficient proficiency in the language of the health survey, for example. Collecting a subset of the corpus of health survey results by inconsistently reliable survey takers can enable analysis and modeling to identify such survey takers early and to improve health surveys to obtain more accurate and reliable results for such survey takers.

Note that the various features of the present invention described above may be practiced alone or in combination. These and other features of the present invention will be described in more detail below in the detailed description of the invention and in conjunction with the following figures.

### A BRIEF DESCRIPTION OF THE DRAWINGS

In order that the present invention may be more clearly ascertained, some embodiments will now be described, by way of example, with reference to the accompanying drawings, in which:

FIG. 1 shows a behavioral health survey analysis system in which a behavioral health survey confidence annotation machine calculates confidence in the reliability of behavioral health survey data in accordance with the present invention;

FIG. 2 is a block diagram of the behavioral health survey confidence annotation machine of FIG. 1 in greater detail;

FIG. 3 is a block diagram of survey annotation logic of the behavioral health survey confidence annotation machine of FIG. 2 in greater detail;

FIG. 4 is a block diagram of confidence annotation logic of the survey annotation logic of FIG. 3 in greater detail;

FIG. 5 is a block diagram of survey annotation system data of the behavioral health survey confidence annotation machine of FIG. 2 in greater detail;

FIG. 6 shows historical behavioral health survey data;

FIG. 7 is a logic flow diagram illustrating a two-pass administration of a behavioral health survey in accordance with the present invention;

FIG. 8 is a logic flow diagram illustrating the measurement of confidence in the reliability of survey data in accordance with the present invention;

FIGS. 9, 10, and 11 are each a logic flow diagram of a respective step of FIG. 8 in greater detail;

FIG. 12 is a block diagram of survey data culling logic of the behavioral health survey confidence annotation machine of FIG. 2 in greater detail;

FIG. 13 is a logic flow diagram illustrating the culling of a corpus of survey taker and survey data by the survey data culling logic of FIG. 12 in accordance with the present invention;

[ 2 3 ] FIG. 14 is a logic flow diagram showing a step of the logic flow diagram of FIG. 13 in greater detail;

FIG. 15 is a logic flow diagram illustrating the identification of highly consistent and highly inconsistent survey takers by the survey data culling logic of FIG. 12 in accordance with the present invention;

FIG. 16 shows a behavioral health survey annotation system in which a behavioral health survey confidence annotation machine, a clinical data server computer system, and a survey taker device cooperate to calculate confidence in the reliability of behavioral health survey data in accordance with the present invention;

FIG. 17 is a logic flow diagram illustrating on-line administration of a behavioral health survey, the real-time annotation of confidence in the reliability of thereof, and associated intervention in the on-line behavioral health survey the survey annotation logic of FIG. 3 in accordance with the present invention; and

FIG. 18 is a block diagram of the behavioral health survey confidence annotation machine of FIG. 1 in greater detail.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

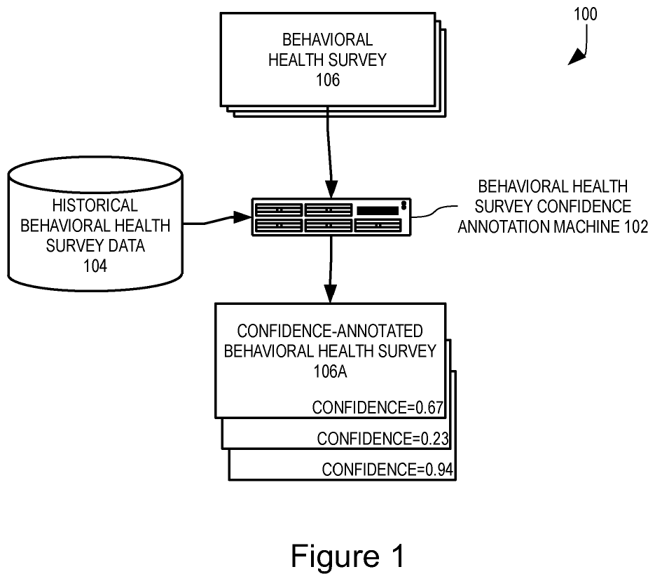

In accordance with the present invention, a behavioral health survey confidence annotation machine 102 (FIG. 1) of a behavioral health survey confidence system 100 determines a degree of confidence in the reliability of a survey taker's responses given in a behavioral health survey. For example, behavioral health survey confidence annotation machine 102 receives behavioral health survey results 106 and determines a degree of confidence in the reliability of the results to produce confidence-annotated behavioral health survey results 106A. Given such a measure of confidence, any analysis of confidence-annotated behavioral health survey results 106A, e.g., statistical analysis and analysis through artificial intelligence (AI) such as deep machine learning, can be significantly more accurate and useful. For example, in such analysis, survey results with lower confidence can be weighted less or disregarded altogether while survey results with higher confidence can be weighted more heavily.

Behavioral health survey confidence annotation machine 102 as described herein can be distributed across multiple computer systems. Distribution of various loads carried by behavioral health survey confidence annotation machine 102 can be distributed among multiple computer systems using conventional techniques.

Identifying unreliable health survey results provides a number of significant advantages. An important one is the culling of health survey results such that a corpus of health survey result data can include only adequately reliable results. Such significantly improves the results of any analysis of the corpus as a whole, including statistical analysis and artificial intelligence (AI) analysis. Any modeling of such a corpus of health survey results can yield much better analysis through removing misleading data labels for statistical inference or when training computational models.

Another significant advantage is that survey takers whose health survey results are consistently unreliable can be identified. The health survey results of these survey takers can be the result of inattentiveness, intent to influence the survey results and indications, illiteracy, or insufficient proficiency in the language of the health survey, for example. Collecting a subset of the corpus of health survey results by inconsistently reliable survey takers can enable analysis and modeling to identify such survey takers early and to improve health surveys to obtain more accurate and reliable results for such survey takers.

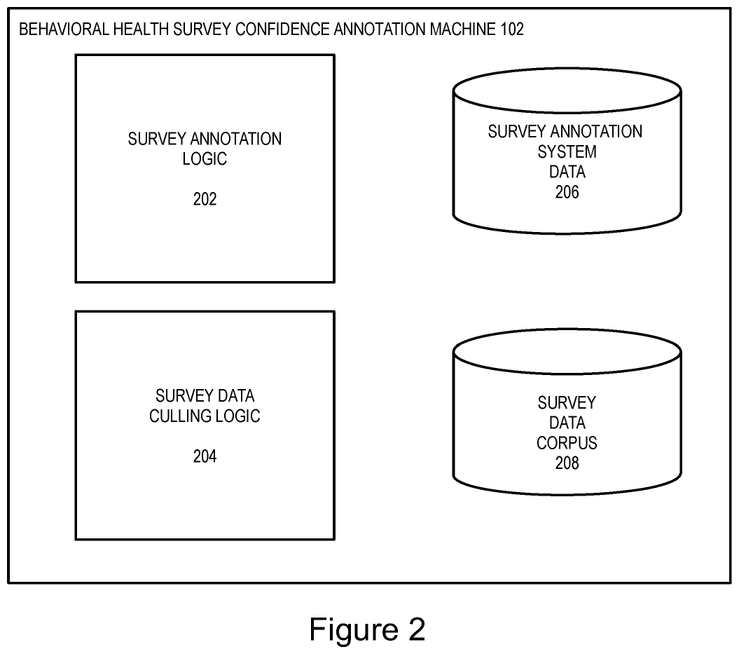

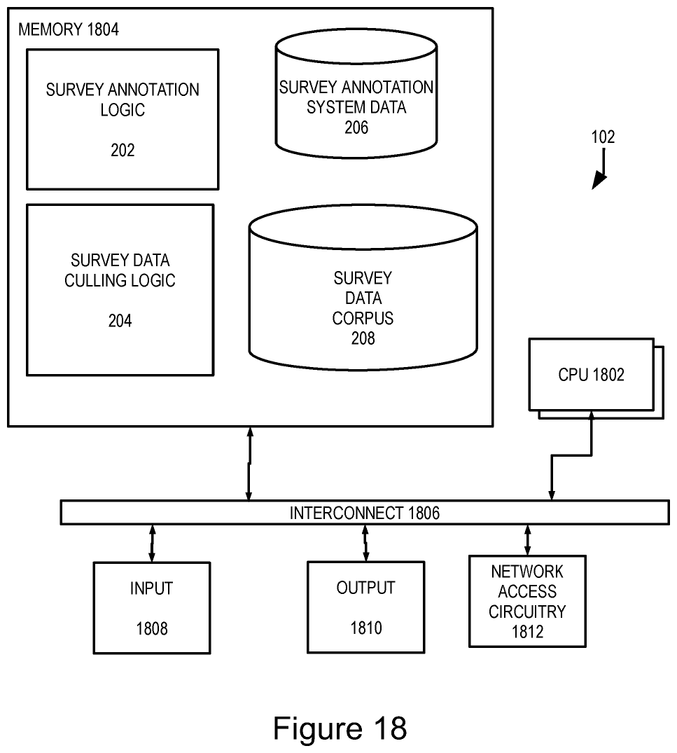

Behavioral health survey confidence annotation machine 102 is shown in greater detail in FIG. 2 and in even greater detail below in FIG. 18. As shown in FIG. 2, behavioral health survey confidence annotation machine 102 includes survey annotation logic 202, survey data culling logic 204, and survey annotation system data 206.

Each of the components of behavioral health survey confidence annotation machine 102 is described more completely below. Briefly, survey annotation logic 202 annotates health survey results confidence levels in the reliability of the results. In an interactive embodiment described below, survey annotation logic 202 also administers an interactive health survey to a human survey taker and annotates confidence levels in the reliability of the responses by the survey taker in real-time and can intervene in the administration of the behavioral health survey to improve quality of survey results. As used herein, reliability of health survey results is the degree to which the results accurately reflect the behavioral health state of the survey taker. Survey data culling logic 204 identifies unreliable behavioral health survey results stored in survey annotation system data 206 and removes those unreliable behavioral health survey results from consideration when analyzing such test results statistically and/or through AI. Such significantly improves such analysis. Survey analysis system data store 210 stores and maintains all survey data needed for, and collected by, analysis in the manner described herein.

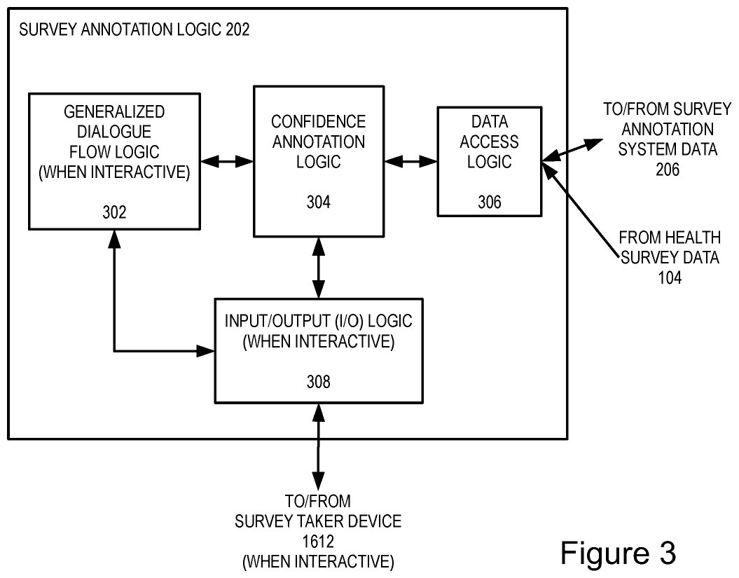

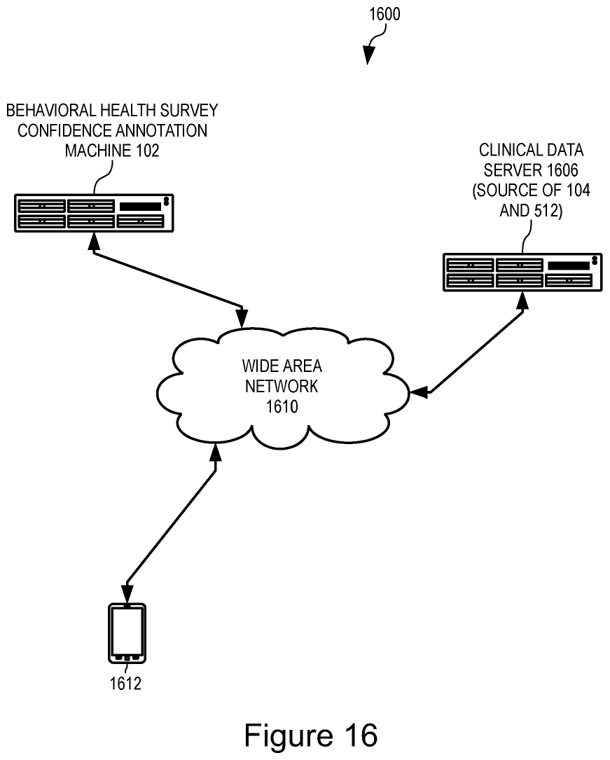

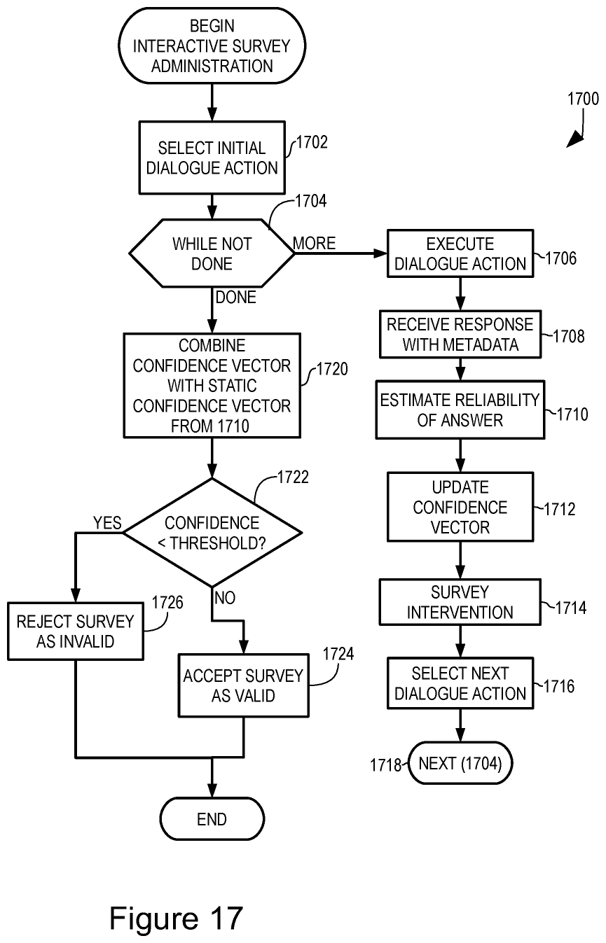

Survey annotation logic 202 is shown in greater detail in FIG. 3. Survey annotation logic 202 includes generalized dialogue flow logic 302, confidence annotation logic 304, data access logic 306, and input/output (I/O) logic 308. Generalized dialogue flow logic 302 and input/output (I/O) logic 308 are used in embodiments in which survey annotation logic 202 administers an interactive health survey with a human survey taker in a manner described below in conjunction with FIGS. 16 and 17 and are described more completely below in conjunction therewith.

Data access logic 306 retrieves data from, and sends data to, survey annotation system data 206 to facilitate operation of survey annotation logic 202.

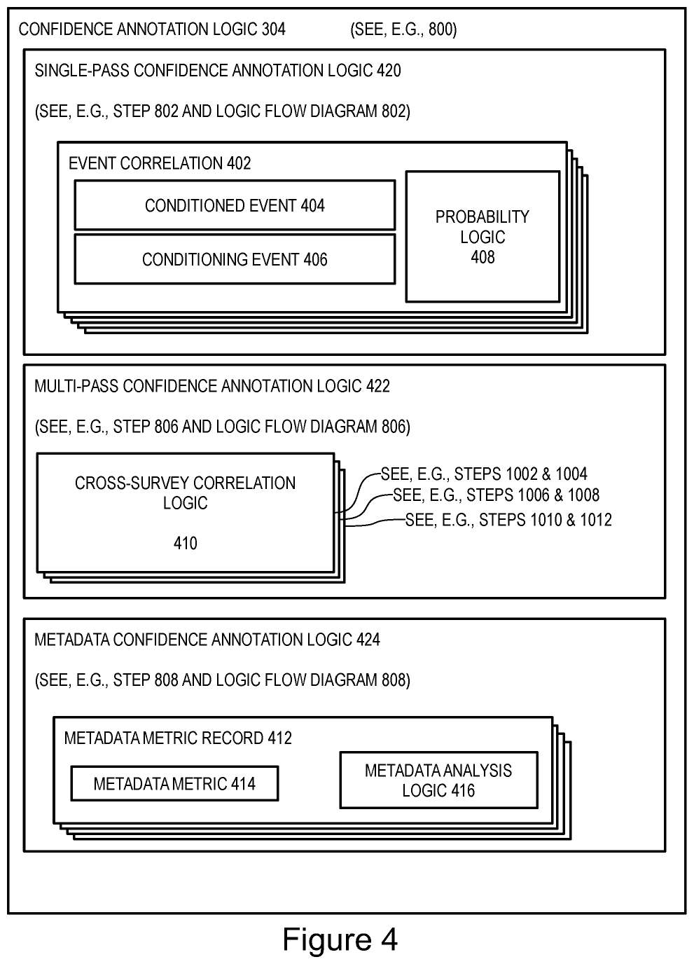

Confidence annotation logic 304 receives survey and survey taker data from survey annotation system data 206 and historical behavioral health survey data 104 through data access logic 306, annotates confidence levels, and stores results of such analysis in survey annotation system data 206 through data access logic 306. Confidence annotation logic 304 is shown in greater detail in FIG. 4.

Confidence annotation logic 304 includes single-pass confidence annotation logic 420, multi-pass confidence annotation logic 422, and metadata confidence annotation logic 424.

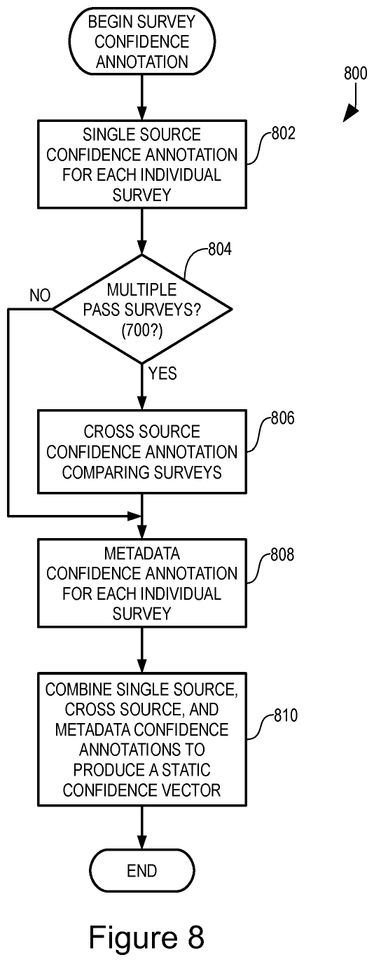

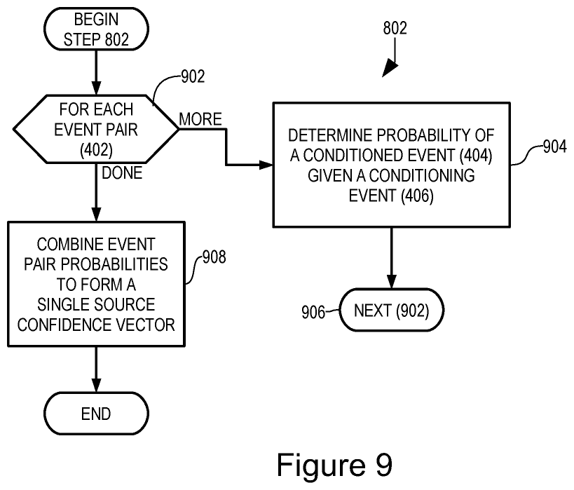

Single-pass confidence annotation logic 420 performs single-pass confidence annotation in a manner described below in conjunction with step 802 (FIG. 8) and logic flow diagram 802 (FIG. 9). Single-pass confidence annotation logic 420 includes a number of event correlations 402, each of which represents a pair of events and facilitates determining of the likelihood of occurrence of a conditioned event 404 given occurrence of a conditioning event 406 for confidence annotation according to probability logic 408.

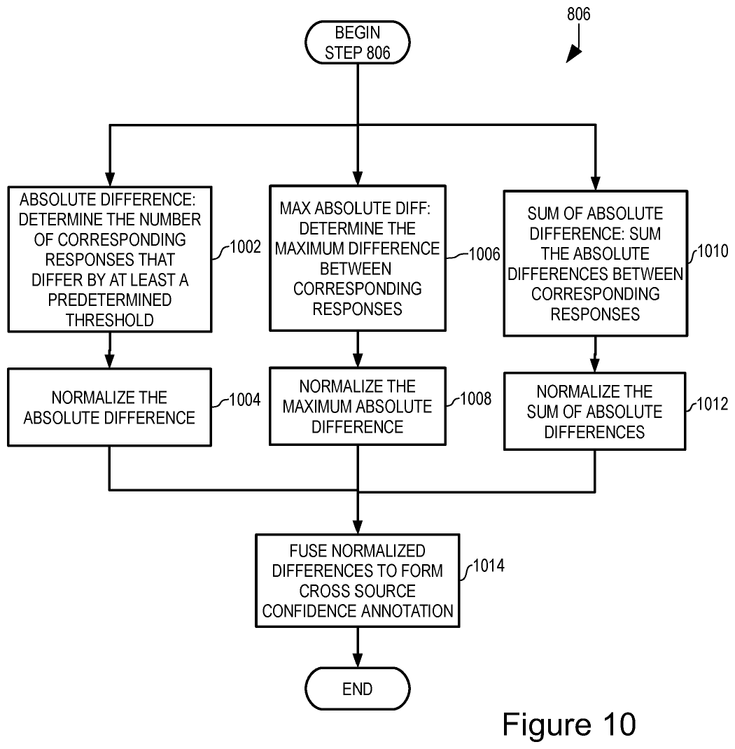

Multi-pass confidence annotation logic 422 performs multi-pass confidence annotation in a manner described below in conjunction with step 806 (FIG. 8) and logic flow diagram 806 (FIG. 10). Multi-pass confidence annotation logic 422 includes multiple approaches for application of cross-survey correlation logic 410 (FIG. 4), each of which represents logic to compare multiple passes of the health survey for confidence annotation.

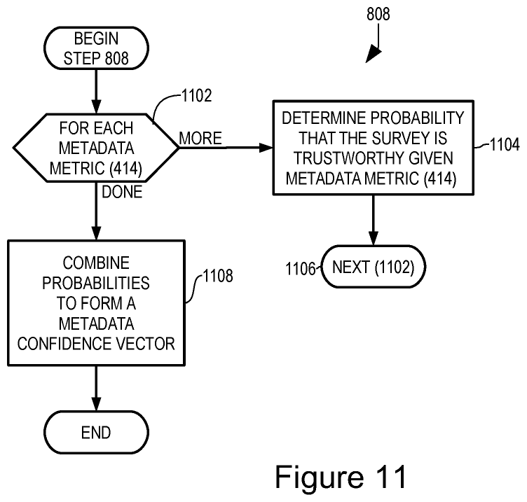

Metadata confidence annotation logic 424 performs metadata confidence annotation in a manner described below in conjunction with step 808 (FIG. 8) and logic flow diagram 802 (FIG. 11). Metadata confidence annotation logic 424 includes metadata metric records 412 (FIG. 4) each of which represents a metadata metric 414 for confidence annotation according to metadata analysis logic 416. Metadata metric 414 can use any portion of survey taker metadata 532 and survey metadata 530, both of which are described below.

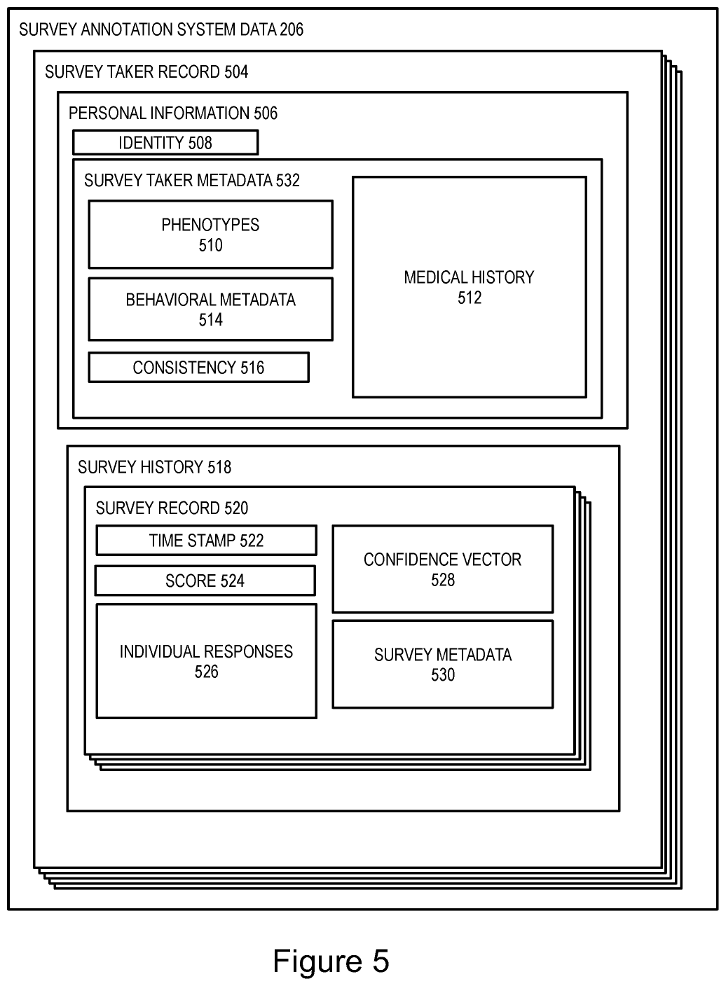

Survey annotation system data 206 (FIG. 2) is shown in greater detail in FIG. 5 and includes a number of survey taker records 504, each of which includes data representing a particular survey taker for which survey annotation logic 202 (FIG. 3) scores confidence in health survey results.

Personal information 506 (FIG. 5) of survey taker record 504 includes data that represents the subject survey taker generally and not specific to any behavioral health surveys. Personal information 506 includes identity 508, which includes data identifying the subject survey taker, and survey taker metadata 532. Personally identifying information is not needed in identity 508 so long as each survey taker can be identified uniquely among all survey takers represented in survey annotation system data 206. Survey taker metadata 532 stores generally any type of information about the survey taker other than identifying data.

For example, phenotypes 510 includes data representing various phenotypes of the subject survey taker. Such phenotypes can include, for example, gender, age (or data of birth), nationality, marital status, income, ethnicity, and language(s) (including a degree of proficiency in each). Medical history 512 includes data representing a medical history of the subject survey taker. Behavioral metadata 514 includes data representing behavior of the user and can include such things as typing speed, reading speed, etc. Consistency 516 includes data representing whether the subject survey taker consistently provides reliable results of health surveys.

Survey history 518 includes data representing prior health surveys, including a number of survey records 520, each of which represents a prior health survey taken by the subject survey taker. Results of a health survey analysis by survey annotation logic 202 are recorded in a survey record 520 as described below.

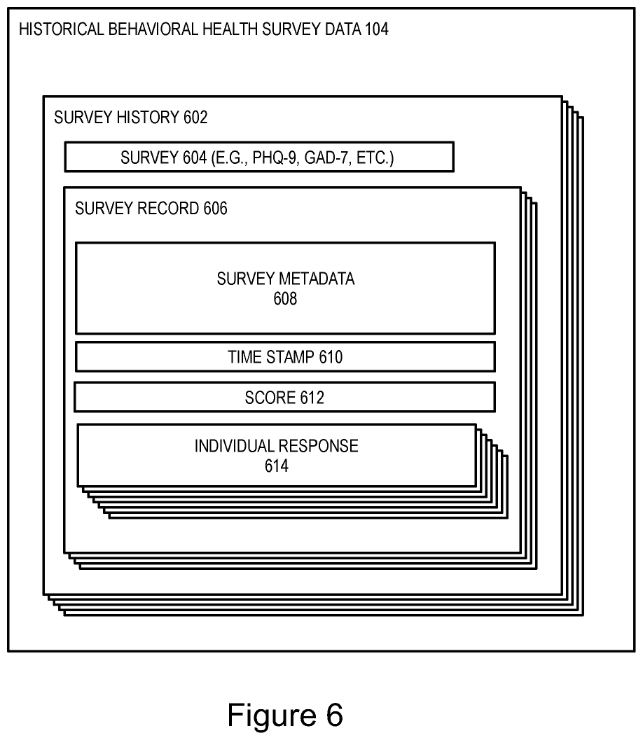

Historical behavioral health survey data 104 is shown in greater detail in FIG. 6. Historical behavioral health survey data 104 represents behavioral survey data available from third-party sources. Accordingly, the particular format and content of historical behavioral health survey data 104 can vary widely from source to source. FIG. 6 represents the general overall nature of available behavioral health survey data to facilitate understanding and appreciation of the present invention.

Historical behavioral health survey data 104 includes a number of survey histories 602, each of which corresponds to a particular type of behavioral health survey, which is identified by survey 604. For example, in a survey history 602 corresponding to the PHQ-9 survey, survey 604 of this particular one of survey histories 602 would identify the PHQ-9 survey.

Each of survey histories 602 includes a number of survey records 606, each of which represents a completed survey of the type identified by survey 604. Survey metadata 608 includes data representing one or more attributes of the subject completed survey that are not represented in other fields of survey record 606. Survey metadata 608 can include information about the particular human taker of the survey, such as the age, gender, and ethnicity of the taker for example. Survey metadata 608 can include other metadata of the survey such as whether and how much compensation was provided to the survey taker and the environment or platform in which the survey was given, for example.

Time stamp 608 represents the date, and can also represent the time, of completion of the subject completed survey. Score 612 represents the overall score of the subject completed survey. Individual responses 614 each represent an individual survey response by the survey taker in the subject completed survey.

It should be appreciated that, since the particular format and content of historical behavioral health survey data 104 can vary widely from source to source, various portions of survey record 606 can be missing, though ordinarily at least score 612 is included. Availability and content of survey metadata 608 varies particularly widely across sources of historical behavioral health survey data 104. It should also be noted that, while surveys represented by survey records 606 are referred to completed surveys, “completed surveys” as used herein are surveys for which the survey taker has ceased taking the survey, even if the survey taker has not responded to all prompts of the survey. Thus, even if the survey taker did not complete responding to all prompts of the survey, administration of the survey to the survey taker has completed.

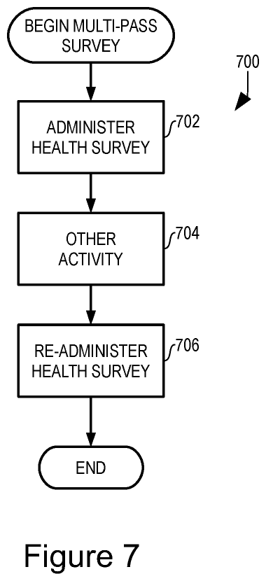

As described above, confidence annotation logic 304 (FIG. 4) includes multi-pass confidence annotation logic 422 for analyzing results of multi-pass health surveys. Multiple-pass health surveys provide especially good insight into the reliability of results of a health survey in ways other techniques don't. Such a multiple-pass health survey is illustrated by logic flow diagram 700 (FIG. 7).

In step 702, the behavioral health survey is administered to the survey taker. The behavioral health survey can be administered by survey annotation logic 202 in the manner described below in conjunction with logic flow diagram 1700 (FIG. 17) or can be administered in a conventional manner. In step 704 (FIG. 7), the survey taker is engaged in activity that is not part of the survey of step 702. This other activity serves to distract the survey taker from perfectly remembering their answers to the first instance of the survey. It can be used for any practical additional purpose such as to gather useful information unrelated to the survey itself. In step 706, the behavioral health survey is administered to the survey taker again. Steps 704 and 706 can be performed by survey annotation logic 202 or by any conventional health survey administration technique. After step 704, this second administration of the behavioral health survey in step 706 is hopefully somewhat of a surprise for the survey taker. Comparison of the two passes can help measure the confidence in the reliability of the survey taker's responses in the manner described below.

Logic flow diagram 800 (FIG. 8) illustrates the measurement of confidence in the reliability of the survey taker's responses in a behavioral health survey after completion of the behavioral health survey by survey annotation logic 202. In step 802, confidence annotation logic 304 evaluates confidence in the reliability of the results of the behavioral health survey for each individual pass of the behavioral health survey. Step 802 is shown in greater detail as logic flow diagram 802 (FIG. 9) and is described below.

In test step 804 (FIG. 8), confidence annotation logic 304 determines whether a multiple-pass behavioral health survey was administered in the manner described above with respect to logic flow diagram 700 (FIG. 7). In this illustrative embodiment, multi-pass behavioral health surveys are not always administered. If a multiple-pass behavioral health survey was administered, confidence annotation logic 304 performs cross-source confidence evaluation by comparing the multiple surveys in step 806 (FIG. 8) as described below in greater detail in conjunction with logic flow diagram 806 (FIG. 10). Conversely, if confidence annotation logic 304 determines that only a single-pass behavioral health survey was administered, confidence annotation logic 304 skips step 806 (FIG. 8).

In step 808, confidence annotation logic 304 uses metadata to evaluate confidence in the reliability of the results of the health survey for each individual pass of the health survey. Step 802 is shown in greater detail as logic flow diagram 802 (FIG. 9) and is described below.

In step 810 (FIG. 8), confidence annotation logic 304 combines the confidence evaluations from steps 802, 806, and 808 to produce a static confidence vector, e.g., confidence vector 528 (FIG. 5). As described below, this static confidence vector can be combined with the intermediate confidence vector in step 1720 (FIG. 17).

Step 802 (FIG. 8) is shown in greater detail as logic flow diagram 802 (FIG. 9). Loop step 902 and next step 906 define a loop in which confidence annotation logic 304 processes each of a number of observed event pairs of the health survey in step 904. Each of the observed event pairs corresponds to a pair of events observed in the survey taker's responses corresponding to conditioned event 404 (FIG. 4) and conditioning event 406 of any of event correlations 402. During each iteration of the loop of steps 902-906 (FIG. 9), the particular one of event correlations 402 processed by confidence annotation logic 304 is sometimes referred to as the subject event correlation.

In step 904, confidence annotation logic 304 determines the probability that conditioned event 404 (FIG. 4) is observed given observation of conditioning event 406 using probability logic 408. For example, suppose conditioned event 404 represents a PHQ-9 score of less than five (5) and conditioning event 406 represents a response of three (3) on the second question of the PHQ-9. The PHQ-9 has nine (9) questions and responses range from zero (0) to three (3), so a score of four (4) or less with a response of three (3) on any question means that at most one other question had a response of one (1) and all other questions had responses of zero (0), which generally has a low probability.

Probability logic 408 determines the probability that conditioned event 404 is observed when conditioning event 406 is also observed. In this illustrative embodiment, probability logic 408 is configured using statistical analysis of an entire corpus of survey data. For example, confidence annotation logic 304 can find all PHQ-9 health surveys with a score of no more than four as represented in score 524 (FIG. 5) of survey history 518 of all survey taker records 504 and/or in score 612 (FIG. 6) of all survey records 606 of historical behavioral health survey data 104 and, from those, determine how many of those have a response of three for the second question as represented in individual responses 526 and/or in individual responses 614. Confidence annotation logic 304 (FIG. 4) can configure probability logic 408 to respond with the ratio of the latter to the former.

Another example of event pairs includes a particular score in the PHQ-9 as conditioned event 404 and a particular score in the GAD-7 as conditioning event 406. In addition to survey history 518 (FIG. 5) of all survey taker records, the data corpus can include health survey scores and responses represented in medical history 512 as well as survey records 606 to the extent that survey taker metadata 608 (FIG. 6) can identify survey records 606, across all survey histories 602, representing completed surveys taken by the same survey taker. In addition, the corpus can be culled in a manner described below to remove unreliable behavioral health survey results from consideration. The corpus is unlikely to change often, so confidence annotation logic 304 (FIG. 4) can update probability logic 408 relatively infrequently, e.g., weekly, monthly, whenever the size of the corpus increases by an appreciable amount (e.g., 2%), or after each culling of the corpus as described below.

Since probability logic 408 is relatively simple as the heavy lifting processing-wise is performed in the configuration of probability logic 408, event correlations 402 can be included in logic within survey taker device 1612 (FIG. 16) such that interactive administration of the health survey with real-time confidence checking in the manner described below with respect to logic flow diagram 1700 (FIG. 17) can be performed by survey taker device 1612 when off-line, i.e., not in communication with behavioral health survey confidence annotation machine 102 through WAN 1610. The same is true for cross-survey correlation logic 410 (FIG. 4) and metadata metric records 412, both of which are described below.

After step 904 (FIG. 9), processing transfers through next step 906 to loop step 902 in which the next observed event pair is processed by confidence annotation logic 304 according to the loop of steps 902-906. When all of the observed event pairs have been processed by confidence annotation logic 304, processing transfers from loop step 902 to step 908.

In step 908, confidence annotation logic 304 combines all probabilities determined in iterative performances of step 904 to form a single-source (e.g., from a single pass of a behavioral health survey) confidence vector. In this illustrative embodiment, confidence annotation logic 304 includes each probability determined in each performance of step 904 as one dimension in the single-source confidence vector.

After step 908, processing according to logic flow diagram 802, and therefore step 802 (FIG. 8), completes.

Step 806 is shown in greater detail as logic flow diagram 806 (FIG. 10). Steps 1002, 1006, and 1010 each correspond to a respective instance of cross-survey correlation logic 410 (FIG. 4). Each cross-survey correlation logic 410 processes at least two (2) passes of the same health survey given contemporaneously in the manner described above with respect to logic flow diagram 700 (FIG. 7).

In step 1002 (FIG. 10), confidence annotation logic 304 determines the number of responses to corresponding prompts that changed by at least a predetermined threshold between the multiple passes using a first instance of cross-survey correlation logic 410 (FIG. 4). For example, confidence annotation logic 304 can determine the number of responses to corresponding prompts that changed by two (2) or more between the multiple passes.

In step 1004 (FIG. 10), confidence annotation logic 304 normalizes the number determined in step 1002 to be a real number in the range of 0.0 to 1.0. Normalization in step 1004 can be accomplished in any of a number of ways. In one illustrative embodiment, confidence annotation logic 304 analyzes the same corpus described above with respect to step 904 (FIG. 9) to determine a percentile for each of the possible results from step 1002 (FIG. 10). If the health survey is the PHQ-9, there are nine (9) possible results. If the health survey is the GAD-7, there are seven (7) possible results. The respective percentiles and the corresponding results from step 1002 can be represented in the first instance of cross-survey correlation logic 410 (FIG. 4) as a simple lookup table.

In step 1006 (FIG. 10), confidence annotation logic 304 determines the greatest absolute difference between responses to corresponding prompts of the multiple passes using a second instance of cross-survey correlation logic 410 (FIG. 4).

In step 1008 (FIG. 10), confidence annotation logic 304 normalizes the number determined in step 1006 to be a real number in the range of 0.0 to 1.0. Normalization in step 1008 can be accomplished in any of a number of ways. In one illustrative embodiment, confidence annotation logic 304 analyzes the same corpus described above with respect to step 904 (FIG. 9) to determine a percentile for each of the possible results from step 1006 (FIG. 10). If the health survey is the PHQ-9, responses range from zero (0) to three (3), so there are four (4) possible results. The respective percentiles and the corresponding results from step 1006 can be represented in the second instance of cross-survey correlation logic 410 (FIG. 4) as a simple lookup table.

In step 1010 (FIG. 10), confidence annotation logic 304 determines the sum of absolute differences between corresponding prompts of the multiple passes using a third instance of cross-survey correlation logic 410 (FIG. 4).

In step 1012 (FIG. 10), confidence annotation logic 304 normalizes the number determined in step 1010 to be a real number in the range of 0.0 to 1.0. Normalization in step 1012 can be accomplished in any of a number of ways. In one illustrative embodiment, confidence annotation logic 304 analyzes the same corpus described above with respect to step 904 (FIG. 9) to determine a percentile for each of the possible results from step 1010 (FIG. 10). Similar to normalization in steps 1004 and 1008, there are a relatively few possible results from step 1010. Accordingly, the respective percentiles and the corresponding results from step 1010 can be represented in the third

In step 1014, confidence annotation logic 304 fuses the normalized values resulting from steps 1004, 1008, and 1012 to form a cross-source confidence vector. In this illustrative embodiment, confidence annotation logic 304 includes each of the normalized values resulting from steps 1004, 1008, and 1012 as one dimension in the cross-source confidence vector. In an alternative embodiment, confidence annotation logic 304 fuses the normalized values resulting from steps 1004, 1008, and 1012 to form a cross-source confidence scalar value by computing a value representative of the normalized values as a whole. Examples of such computing include, for example, weighted linear and nonlinear combination including statistical voting, local regression, simple regression, and so on.

After step 1014, processing according to logic flow diagram 806, and therefore step 806 (FIG. 8), completes.

Step 808 is shown in greater detail as logic flow diagram 808 (FIG. 11). Loop step 1102 and next step 1106 define a loop in which confidence annotation logic 304 processes each of metadata metric records 412 (FIG. 4) in step 1104. During each iteration of the loop of steps 1102-1106 (FIG. 11), the particular one of metadata metric records 412 (FIG. 4) processed by confidence annotation logic 304 is sometimes referred to as the subject metadata metric record.

In step 1104 (FIG. 11), confidence annotation logic 304 determines the probability that the subject health survey results are reliable according to metadata metric 414 (FIG. 4) of the subject metadata metric record using probability logic 416 of the subject metadata metric record. For example, suppose metadata metric 414 represents the duration of the survey taker's delay before responding to the first prompt of the health survey. It has been observed that the longer initial delay, the more reliable the results of the health survey, perhaps representing greater consideration of the behavioral health survey before beginning to respond. Accordingly, metadata analysis logic 416 of the same metadata metric record 412 scores greater confidence that the results of the behavioral health survey are reliable when the initial delay is longer.

Metadata analysis logic 416 determines the probability that the subject behavioral health survey results are reliable according to metadata metric 414 of the subject metadata metric record. In this illustrative embodiment, metadata analysis logic 416 is configured using statistical analysis of the same corpus described above with respect to step 904 (FIG. 9). For example, confidence annotation logic 304 (FIG. 4) can find all health surveys of survey history 518 (FIG. 5) of all survey taker records 504 and correlate confidence vectors 528 with the length of the initial delay as presented in survey metadata 530. Confidence annotation logic 304 (FIG. 4) can also use the same data from survey metadata 608 (FIG. 6) to the extent such data is available.

There are numerous other illustrative examples of metadata metrics that can be represented by metadata metric 414 including the following. Delays prior to responding to other prompts of the health survey as well as the overall duration of the health survey can be metadata metrics. It has been observed that longer and more varied delays in responding to the various prompts, as well as longer test durations, indicate more reliable results of the health survey, suggesting more deliberately considered responses. The number of corrections made by the survey taker to previously given responses also indicates a more deliberate consideration of the behavioral health survey. Deviations in the order of responses given by the survey taker from the order in which the prompts are presented to the survey taker similarly indicates greater attention and careful consideration. In embodiments in which survey taker device 1612 (FIG. 16) captures audio and/or video signals of the survey taker during administration of the behavioral health survey, such signals can be analyzed for gaze and eye tracking as well as analysis of vocal responses to the prompts. Metadata analysis logic 416 (FIG. 4) can also analyze metadata metric 414 in the context of the survey taker's behavior when not taking the health survey, e.g., using extra-survey metadata 514 (FIG. 5) and, to the extent such data is available, survey metadata 608 (FIG. 6).

In addition to user interface metadata, confidence annotation logic 304 (FIG. 4) can use survey taker metadata, e.g., as represented in phenotypes 510 (FIG. 5) and/or medical history 512, as metrics to analyze confidence in the reliability of responses of a given behavioral health survey. Just a few illustrative examples of the possible survey taker metadata metrics include age, gender, ethnicity, location, time of day, collection platform, electronic medical record (EMR) data, claims data, survey taker history, and past survey scores.

After step 1104 (FIG. 11), processing transfers through next step 1106 to loop step 1102 in which the next observed event pair is processed by confidence annotation logic 304 according to the loop of steps 1102-1106. When all of the metadata metric records have been processed by confidence annotation logic 304, processing transfers from loop step 1102 to step 1108.

In step 1108, confidence annotation logic 304 combines all probabilities determined in iterative performances of step 1104 to form a metadata confidence vector. In this illustrative embodiment, confidence annotation logic 304 includes each probability determined in each performance of step 1104 as one dimension in the metadata confidence vector.

After step 1108, processing according to logic flow diagram 808, and therefore step 808 (FIG. 8), completes.

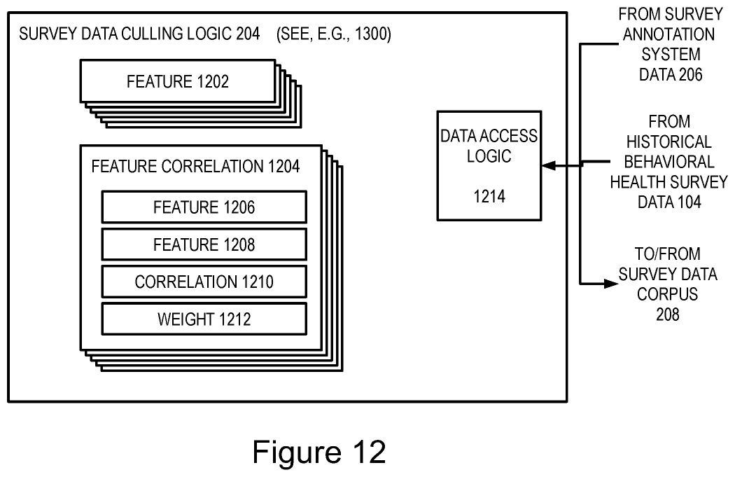

As described above, survey data culling logic 204 identifies low-confidence behavioral health survey results stored in survey data corpus 208 and removes those low-confidence health survey results from consideration when analyzing such survey results statistically and/or through AI. Survey data corpus 208 represents that portion of historical behavioral health survey data 104 (FIG. 6) and survey annotation system data 206 (FIG. 5) used by survey annotation logic 202 in the manner described above. Survey data corpus 208 can include copies of portions of historical behavioral health survey data 104 and survey annotation system data 206 and/or can include such data by reference thereto. Survey data culling logic 204 is shown in greater detail in FIG. 12.

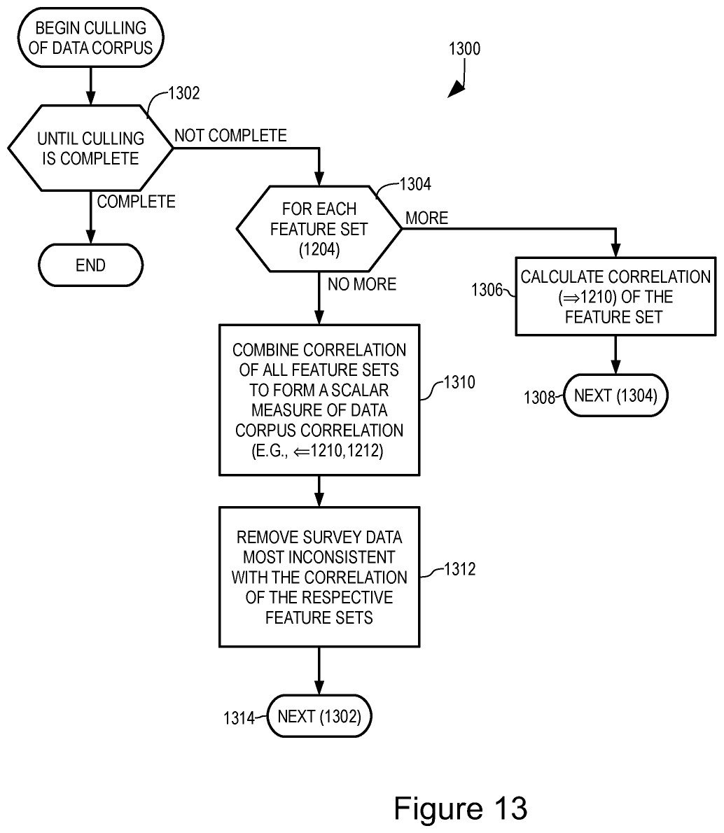

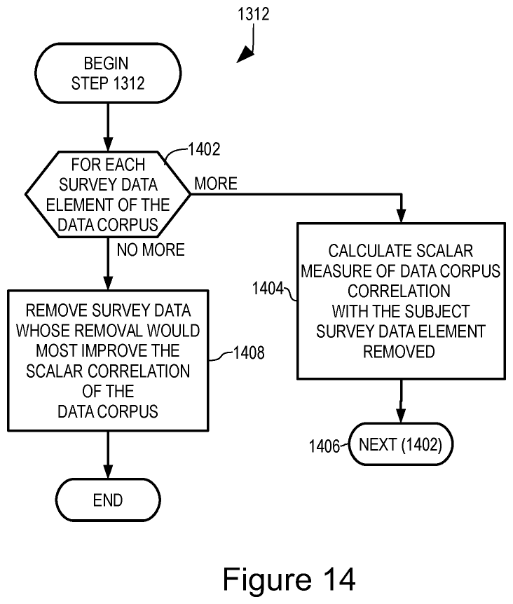

Survey data culling logic 204 includes a number of features 1202, a number of feature correlations 1204, and data access logic 1214. Data access logic 1214 retrieves data from, and sends data to, survey annotation system data 206 to facilitate operation of survey data culling logic 204. Each of features 1202 represents any item of information in survey taker records 504. Features 1202 are selected in a manner described more completely below. Feature correlations 1204 represent sets of two or more of features 1202 and are described more completely below in the context of logic flow diagram 1300 (FIG. 13). In this illustrative embodiment, feature correlations 1204 represent pairs of features 1202.

The manner in which survey data culling logic 204 (FIG. 2) culls survey data corpus 208 is illustrated in logic flow diagram 1300 (FIG. 13). Loop step 1302 and next step 1314 define a loop in which survey data culling logic 204 iteratively culls survey data corpus 208 according to steps 1304-1312 until the culling is deemed complete. In one embodiment, survey data culling logic 204 deems culling to be complete when the overall measure of confidence in the reliability of all health survey results of survey data corpus 208 is at least a predetermined minimum threshold. In an alternative embodiment, survey data culling logic 204 deems culling to be incomplete as long as the size of survey data corpus 208 is above a predetermined statistically acceptable size. In yet another embodiment, survey data culling logic 204 deems culling to be incomplete when further iterations of the loop of steps 1302-1314 fail to significantly improve the overall measure of confidence in the reliability of all health survey results of survey data corpus 208. Until culling is complete, processing transfers from loop step 1302 to loop step 1304.

Loop step 1304 and next step 1308 define a loop in which survey data culling logic 204 processes each of feature correlations 1204 (FIG. 12) according to step 1306 (FIG. 13). During a given iteration of the loop of steps 1304-1308, the particular one of feature correlations 1204 processed by survey data culling logic 204 is sometimes referred to as the subject feature correlation.

In step 1306, survey data culling logic 204 calculates correlation 1210 (FIG. 12) for the features of the subject feature correlation. Correlation 1210 can be any type of measurement of relationships between two or more of features 1202. Examples of such relationship measurements include correlation, mutual information, and measurements based on neural-network models such as graphical models. In this illustrative embodiment, correlation 1210 represents mutual information of two features, i.e., features 1206 and 1208. Feature 1206 identifies one of features 1202, and feature 1208 identifies a different one of features 1202. In this illustrative embodiment, a feature correlation 1204 exists for each and every unique combination of features 1202. Survey data culling logic 204 stores the calculated correlation in correlation 1210 of the subject feature correlation.

After calculating correlation 1210 for the features of the set of the subject feature correlation in step 1306 (FIG. 13), processing by survey data culling logic 204 transfers through next step 1308 to loop step 1304 and survey data culling logic 204 processes the next of feature correlations 1204 according to the loop of steps 1304-1308. When all of feature correlations 1204 have been processed, processing transfers from loop step 1304 to step 1310.

In step 1310 (FIG. 13), survey data culling logic 204 combines all correlations 1210 (FIG. 12) to form a scalar measure of data quality of survey data corpus 208. In this illustrative embodiment, survey data culling logic 204 combines all correlations 1210 by calculating a weighted mean in which each correlation 1210 is weighted by a corresponding weight 1212. Weights 1212 are configured in a manner described more completely below.

In step 1312 (FIG. 13), survey data culling logic 204 removes from survey data corpus 208 survey data that is most inconsistent with the correlation calculated in step 1306. Step 1312 is shown in greater detail as logic flow diagram 1312 (FIG. 14).

Loop step 1402 and next step 1406 define a loop in which survey data culling logic 204 processes each survey data element according to step 1404. In this illustrative embodiment, survey data elements are each a survey record 520 (FIG. 5). During a given iteration of the loop of steps 1402-1406, the particular survey record 520 processed by survey data culling logic 204 is sometimes referred to as the subject survey record.

In step 1404 (FIG. 14), survey data culling logic 204 calculates the scalar measure of data corpus correlation in the manner described above with respect to step 1310 (FIG. 13) but excludes the subject survey record from the calculation. In effect, survey data culling logic 204 removes the subject survey record from the scalar measure of correlation of the data corpus to calculate what the scalar measure of data corpus correlation would be if the subject survey record were removed from survey data corpus 208.

After step 1404 (FIG. 14), processing by survey data culling logic 204 transfers through next step 1406 to loop step 1402 in which the next survey data element is processed by survey data culling logic 204 according to the loop of steps 1402-1406. When all survey data elements have been processed by survey data culling logic 204, processing transfers from loop steps 1402 to step 1408.

In step 1408, survey data culling logic 204 removes the survey data elements whose removal would most improve the scalar measure of correlation of survey data corpus 208. In particular, survey data culling logic 204 ranks the survey data elements according to their respective scalar measures of data corpus correlation as calculated in step 1404 and removes from survey data corpus 208 those survey data elements with the highest scalar measures calculated in step 1404.

After step 1408, logic flow diagram 1312, and therefore step 1312 (FIG. 13), completes. From step 1312, processing by survey data culling logic 204 transfers through next step 1314 to loop step 1302 and steps 1304-1312 are repeated until survey data culling logic 204 determines that culling is complete. When culling is complete, processing according to logic flow diagram 1300 completes.

Features 1202 (FIG. 12) and weights 1212 are selected in a manner to measure correlation one would expect in a data corpus with high confidence in the reliability of survey taker responses to behavioral health survey prompts. Such a data corpus is sometimes referred to as a high quality data corpus. Features 1202 can be selected manually by human scientists skilled in behavioral health surveys and/or data science. Inspiration for selecting features 1202 can come from the scientist's intuition or scientific literature reporting correlations between features and results of health surveys. By performing statistical analysis of survey data corpus 208 with respect to the selected features, the scientist can identify features that correlate relatively strongly with survey data quality. In effect, features 1202 can be selected by trial and error.

Such trial and error can be automated. Survey data culling logic 204 can be configured to perform statistical analysis of various data fields of survey taker record 504 (FIG. 5) with respect to the data corpus to identify data fields with strong correlation with survey data quality. Data fields with such strong correlations can be some of features 1202 (FIG. 12).

Weights 1212 correlate to an expected mutual information 1210. In other words, feature correlations 1204 whose correlation 1210 is expected to be relatively high in a high quality data corpus are attributed a greater weight 1212. As with features 1202, weights 1212 can be improved by trial and error such that the scalar measure of data quality of the data corpus more accurately represents the quality of the data corpus.

Thus, by removing low-quality survey data from survey data corpus 208, survey data culling logic 204 significantly improves the quality of survey data corpus 208 and, as a result, any statistical or AI analysis of survey data corpus 208. For example, the higher-quality data corpus significantly improves the measuring of confidence in the reliability of responses to health surveys in the manner described above.

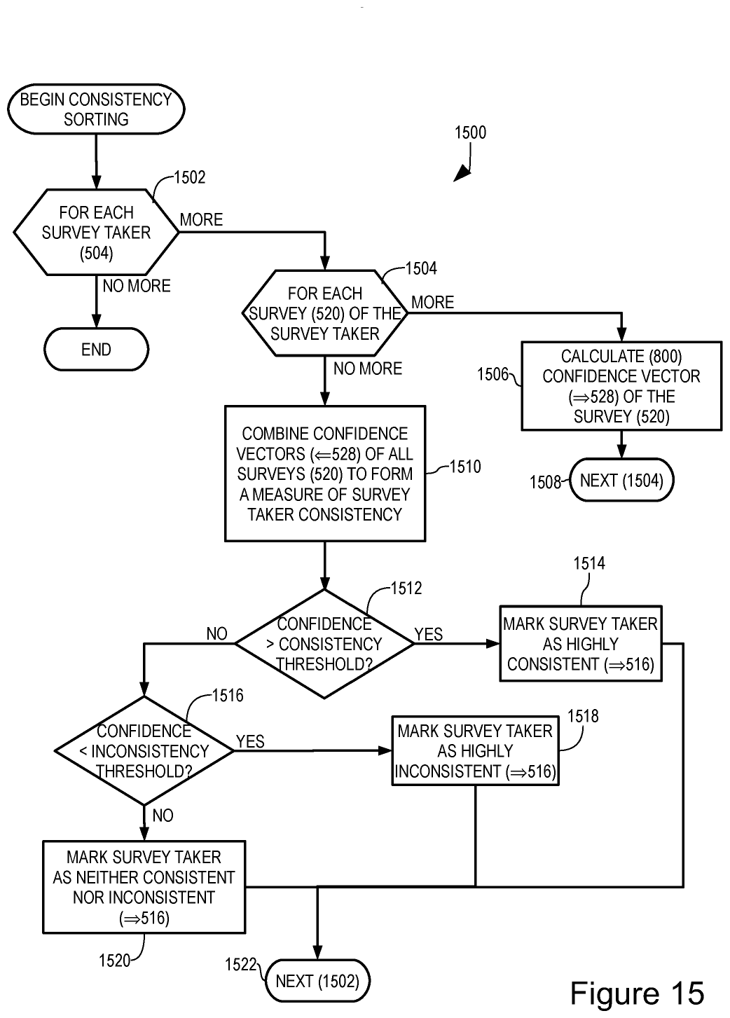

Survey data culling logic 204 (FIG. 2) identifies survey takers whose survey results are highly consistently reliable and survey takers whose survey results reliability is highly inconsistent in a manner illustrated by logic flow diagram 1500 (FIG. 15).

Loop step 1502 and next step 1522 define a loop in which survey data culling logic 204 processes each of survey taker records 504 (FIG. 5) according to steps 1504-1520 (FIG. 15). During an iteration of the loop of steps 1502-1522, the particular one of survey taker records 504 (FIG. 5) processed by survey data culling logic 204 is sometimes referred to as the subject survey taker record, and the survey taker represented by the subject survey taker record is sometimes referred to as the subject survey taker.

Loop step 1504 and next step 1508 define a loop in which survey data culling logic 204 processes each of survey records 520 (FIG. 5) of the subject survey taker record according to step 1506 (FIG. 15). During an iteration of the loop of steps 1504-1508, the particular one of survey records 520 (FIG. 5) processed by survey data culling logic 204 is sometimes referred to as the subject survey record.

In step 1506 (FIG. 15), survey data culling logic 204 calculates a confidence vector representing a degree of confidence in the reliability of the results of the subject survey record in the manner described above with respect to logic flow diagram 800 (FIG. 8). After step 1506, processing transfers through next step 1508 to loop step 1504 and survey data culling logic 204 processes the next of survey records 520 of the subject survey taker record. When all survey records 520 of the subject survey taker record have been processed according to the loop of steps 1504-1508, processing by survey data culling logic 204 transfers to step 1510.

In step 1510, survey data culling logic 204 combines confidence vectors 528 calculated in the loop of steps 1504-1508 to form a single measure of consistency of the responses of the subject survey taker. Examples of such combination include, for example, weighted linear and nonlinear combination, local regression, simple regression, and mathematical voting.

In test step 1512, survey data culling logic 204 compares the single measure of consistency of the responses of the subject survey taker to a predetermined high consistency threshold. If the single measure of consistency is greater than the predetermined high consistency threshold, survey data culling logic 204 marks the subject survey taker as highly consistent by storing data so indicating in consistency 516 (FIG. 5) of the subject survey taker record in step 1514 (FIG. 15). Conversely, if the single measure of consistency is not greater than the predetermined high consistency threshold, processing transfers to test step 1516.

In test step 1516, survey data culling logic 204 compares the single measure of consistency of the responses of the subject survey taker to a predetermined high inconsistency threshold. If the single measure of consistency is less than the predetermined high inconsistency threshold, survey data culling logic 204 marks the subject survey taker as highly inconsistent by storing data so indicating in consistency 516 (FIG. 5) of the subject survey taker record in step 1518 (FIG. 15). Conversely, if the single measure of consistency is not less than the predetermined high inconsistency threshold, survey data culling logic 204 marks the subject survey taker as neither highly consistent nor highly inconsistent by storing data so indicating in consistency 516 (FIG. 5) of the subject survey taker record in step 1520 (FIG. 15).

From any of steps 1514, 1518, and 1520, processing by survey data culling logic 204 transfers through next step 1522 to loop step 1502 and survey data culling logic 204 processes the next of survey taker records 504 (FIG. 5) according to the loop of steps 1502-1522. It should be appreciate that, while steps 1512-1520 show an illustrative embodiment in which there are three (3) categories of survey taker consistency, alternative embodiments can sort survey takers into fewer or more categories or simply store the result of step 1510 to annotate survey taker record 504 ((5) with consistency 516, allowing custom consistency filters to be constructed for particular purposes of the users of survey annotation system data 206. When all of survey taker records 504 have been processed, processing according to logic flow diagram 1500 completes.

Thus, survey data culling logic 204 identifies survey takers who tend to be highly consistent in their survey responses and those who tend to be highly inconsistent. Knowing whether a given survey taker tends to be consistently reliable can be useful in both (i) annotating confidence levels in survey results from the survey taker and (ii) culling survey data in the manner described above. For example, metadata confidence annotation logic 424 (FIG. 4) can include a metadata metric record 412 that has consistency 516 (FIG. 5) as its metadata metric 414 and metadata analysis logic 416 can estimate a probability that survey results are reliable from consistency 516. Such would cause consistency 516 to influence metadata confidence evaluation in step 808 and therefore the static confidence vector produced in step 810. In addition, since confidence vectors can be used in the manner described below to influence administration of, and processing of results of, a behavioral health survey, consistency 516 influences such administration and processing.

Moreover, identifying a significant number of survey takers whose survey results are highly inconsistent can be very helpful in improving behavioral surveys themselves or at least their administration to survey takers. In particular, statistical and/or AI analysis of survey taker records 504 for survey takers marked as highly inconsistent can identify aspects of behavioral health surveys that fail to elicit more reliable results. Correcting such aspects can significantly improve the reliability of behavioral health surveys overall.

As described above, behavioral health survey confidence annotation machine 102 can interactively administer behavioral health surveys to survey takers. A behavioral health survey confidence system 1600 (FIG. 16) in which behavioral health survey confidence annotation machine 102 interactively administers behavioral health surveys to survey takers is shown in FIG. 16. Historical behavioral health survey data 104 (FIG. 1) is available from a number of clinical data servers such as clinical data server 1606 (FIG. 16) though a wide area network (WAN) 1610, that is the Internet in this illustrative embodiment. Behavioral health survey confidence annotation machine 102 acts as a server computer system that administers the health survey with the survey taker through survey taker device 1612 through WAN 1610 and determines confidence level annotation in the manner described above.

As described briefly above, survey annotation logic 202 (FIG. 3) includes generalized dialogue flow logic 302 and I/O logic 308. I/O logic 308 effects administration of the health survey by sending prompt data to, and receiving responsive data from, survey taker device 1612. Some or all of the prompt data causes survey taker device 1612 to present prompts to the survey taker. For example, if the health survey is a form of the PHQ-9 health survey, the prompt data causes survey taker device 1612 to present the various questions of the PHQ-9 test to the survey taker. The responsive data represents responses by the survey taker to the presented prompts. The responsive data can also include metadata such as user interface data as described more completely above. I/O logic 308 receives data from generalized dialogue flow logic 302 that specifies prompts to be presented to the survey taker and sends prompt data representing those prompts to survey taker device 1612.

Generalized dialogue flow logic 302 conducts the health survey with the human survey taker by determining what prompts I/O logic 308 should present to the survey taker and receives the response data from I/O logic 308 to determine (i) which prompt should be presented next, if any, and (ii) when the behavioral health survey is complete.

In this illustrative embodiment, survey annotation logic 202 administers a health survey in the manner illustrated in logic flow diagram 1700 (FIG. 6). It should be appreciated that some of the steps of logic flow diagram 1700 can be performed by logic within survey taker device 1612 as described above. In step 1702, generalized dialogue flow logic 302 (FIG. 3) selects a prompt or other dialogue action to initiate the dialogue with the survey taker.

Loop step 1704 (FIG. 17) and next step 1718 define a loop in which generalized dialogue flow logic 302 conducts the behavioral health survey according to steps 1706-1716 until generalized dialogue flow logic 302 determines that the behavioral health survey is completed.

In step 1706 (FIG. 17), generalized dialogue flow logic 302 (FIG. 3) causes I/O logic 308 to carry out the dialogue action selected in step 1702 or in the most recent performance of step 1716 described below. If the selected dialogue behavior is to present the next prompt of the current behavioral health survey, generalized dialogue flow logic 302 sends prompt data representing the selected prompt to survey taker device 1612 (FIG. 16) so as to cause survey taker device 1612 to present the selected prompt to the survey taker.

In step 1708, generalized dialogue flow logic 302 (FIG. 3) receives response data representing the survey taker's response to the prompt and user interface metadata associated with the response data. In addition, survey annotation logic 202 causes, through data access logic 306, survey annotation system data 206 to include the received response in individual responses 526 (FIG. 5), and the received user interface metadata in survey metadata 530, of survey record 520 representing the current, in-progress behavioral health survey.

In step 1710 (FIG. 17), confidence annotation logic 304 (FIG. 3) determines a degree of confidence in the reliability of the survey taker's response in a manner described above with respect to logic flow diagram 800 (FIG. 8). In step 1712 (FIG. 17), confidence annotation logic 304 updates confidence vector 528 (FIG. 5) of survey record 520 representing the current, in-progress behavioral health survey such that confidence vector 528 represents an intermediate measure of confidence in the reliability of the survey taker's responses for the health survey so far.

In step 1714 (FIG. 17), confidence annotation logic 304 performs survey intervention analysis to determine whether intervention in the currently administered behavioral health survey is warranted. Using the updated, intermediate confidence vector determined in step 1712, or a portion thereof, confidence annotation logic 304 determines whether a dialogue action other than presenting the next prompt of the behavioral health survey is warranted. If the intermediate confidence vector is particularly low, confidence annotation logic 304 can determine that the next dialogue action is to terminate the behavioral health survey. If the intermediate confidence vector is somewhat low or portions of the intermediate confidence vector, particularly those pertaining to user interface metadata, indicate that the survey taker is rushing through the behavioral health survey without given careful consideration to the prompts of the survey, confidence annotation logic 304 can select a next dialogue action that is designed to slow the survey taker down and give more careful consideration to the prompts of the behavioral health survey. Examples of such dialogue actions can include, for example, asking the survey taker whether it is currently a convenient time to take the survey and even scheduling the survey taker to take the survey at a later time, prompting the survey taker to slow down, asking the survey taker to confirm a previously given response, causing the survey to be administered again as a multiple-pass behavioral health survey as described above, and pausing between presentation of survey prompts to the survey taker.

In step 1716, generalized dialogue flow logic 302 (FIG. 3) selects the next dialogue action to be carried out in administration of the current behavioral health survey. Ordinarily, unless confidence annotation logic 304 determines that a particular dialogue action is to be taken next, the next dialogue action will be the next prompt to be presented to the subject survey taker in the next performance of step 1706 (FIG. 17). Processing transfers through next step 1718 to loop step 1704.

Generalized dialogue flow logic 302 (FIG. 3) repeats the loop of steps 1704-1718 until generalized dialogue flow logic 302 determines that the behavioral health survey is complete and processing transfers from loop step 1704 to step 1720.

In step 1720, confidence annotation logic 304 determines a static confidence vector from the entirety of the results received in iterative performances of step 1708 in the manner described above in conjunction with logic flow diagram 800 (FIG. 8) and combines the static confidence vector with the intermediate confidence vector resulting from step 1712 (FIG. 17) and stores the result of the combination in confidence vector 528 (FIG. 5).

In test step 1722 (FIG. 17), confidence annotation logic 304 determines whether confidence vector 528 (FIG. 5) represents a measure of confidence that is below a predetermined threshold. If not, confidence annotation logic 304 accepts the results of the health survey as valid in step 1724 and completes survey record 520 representing the current health survey by (i) recording the current date and time in time stamp 522 and (ii) determining and storing in score 524 a final score of the health survey in accordance with the received responses.

If confidence annotation logic 304 determines whether confidence vector 528 (FIG. 5) represents a measure of confidence that is below the predetermined threshold in test step 1722, confidence annotation logic 304 rejects the current health survey as invalid in step 1726 (FIG. 17). In some embodiments, confidence annotation logic 304 rejects the current health survey as invalid by discarding survey record 520 representing the current health survey. In other, alternative embodiments, confidence annotation logic 304 rejects the current health survey as invalid by so marking survey record 520 representing the current health survey.

After step 1724 or step 1726, processing according to logic flow diagram 1700 completes. Thus, behavioral health survey confidence annotation machine 102 estimates a measure of confidence in the reliability of behavioral health survey results and can even terminate the behavioral health survey early upon determining that the confidence is below a predetermined threshold.

In some embodiments, survey annotation logic 202 can be implemented in survey taker device 1612 (FIG. 16) such that interactive administration of a behavioral health survey in the manner described in conjunction with logic flow diagram 1700 (FIG. 17) can be performed by survey taker device 1612 when offline, i.e., when not in communication with behavioral health survey confidence annotation machine 102. As described above, probability logic 408, cross-survey correlation logic 410, and metadata analysis can be simplified and only periodically updated such that survey annotation logic 202 can be implemented in a device with significantly less processing resources than behavioral health survey confidence annotation machine 102, e.g., survey taker device 1612.

Behavioral health survey confidence annotation machine 102 is shown in greater detail in FIG. 18. As noted above, it should be appreciated that the behavior of behavioral health survey confidence annotation machine 102 described herein can be distributed across multiple computer systems using conventional distributed processing techniques. Behavioral health survey confidence annotation machine 102 includes one or more microprocessors 1802 (collectively referred to as CPU 1802) that retrieve data and/or instructions from memory 1804 and execute the retrieved instructions in a conventional manner. Memory 1804 can include generally any computer-readable medium including, for example, persistent memory such as magnetic and/or optical disks, ROM, and PROM and volatile memory such as RAM.

CPU 1802 and memory 1804 are connected to one another through a conventional interconnect 1806, which is a bus in this illustrative embodiment and which connects CPU 1802 and memory 1804 to one or more input devices 1808, output devices 1810, and network access circuitry 1812. Input devices 1808 generate signals in response to physical manipulation by a human user and can include, for example, a keyboard, a keypad, a touch-sensitive screen, a mouse, a microphone, and one or more cameras. Output devices 1810 can include, for example, a display—such as a liquid crystal display (LCD)—and one or more loudspeakers. Network access circuitry 1812 sends and receives data through computer networks such as WAN 1610 (FIG. 16). Server computer systems often exclude input and output devices, relying instead on human user interaction through network access circuity. Accordingly, in some embodiments, behavioral health survey confidence annotation machine 102 does not include input devices 1808 and output devices 1810.

A number of components of behavioral health survey confidence annotation machine 102 are stored in memory 1804. In particular, survey annotation logic 202 and survey data culling logic 204 are each all or part of one or more computer processes executing within CPU 1802 from memory 1804 As used herein, “logic” refers to (i) logic implemented as computer instructions and/or data within one or more computer processes and/or (ii) logic implemented in electronic circuitry.

Survey annotation system data 206 and survey data corpus 208 are each data stored persistently in memory 1804 and can be implemented as all or part of one or more databases.

It should be appreciated that the distinction between servers and clients is largely an arbitrary one to facilitate human understanding of purpose of a given computer. As used herein, “server” and “client” are primarily labels to assist human categorization and understanding.

Moreover, many modifications of and/or additions to the above described embodiment(s) are possible. For example, with patient consent, corroborative patient data for mental illness diagnostics can be extracted from one or more of the patient's biometrics including heart rate, blood pressure, respiration, perspiration, body temperature. It may also be possible to use audio without words, for privacy or for cross-language analysis. It is also possible to use acoustics modeling without visual cues. Although sub-section titles have been provided to aid in the description of the invention, these titles are merely illustrative and are not intended to limit the scope of the present invention. In addition, where claim limitations have been identified, for example, by a numeral or letter, they are not intended to imply any specific sequence.

The present invention is defined solely by the claims which follow and their full range of equivalents. It is intended that the following appended claims be interpreted as including all such alterations, modifications, permutations, and substitute equivalents as fall within the true spirit and scope of the present invention.

1.-98. (canceled)

99. A method for processing survey responses, comprising:
(a) obtaining, during a first session, (i) a first plurality of responses to a plurality of queries in a survey and (ii) first metadata associated with said first plurality of responses, which first metadata comprises a plurality of first response times for said first plurality of responses;
(b) obtaining, during a second session, (i) a second plurality of responses to said plurality of queries and (ii) second metadata associated with said second plurality of responses, which second metadata comprises a plurality of second response times for said second plurality of responses; and
(c) processing (i) said first plurality of responses and said second plurality of responses or (ii) said first metadata and said second metadata, to identify variation, wherein said variation is indicative of a reliability of said first plurality of responses.

100. The method of claim 99, wherein said reliability of said first plurality of responses is determined based on said variation between said first metadata and said second metadata, and wherein (c) further comprises determining whether said variation between said first metadata and said second metadata exceeds a variation threshold.

101. The method of claim 100, wherein determining whether said variation between said first metadata and said second metadata exceeds said variation threshold comprises determining whether an aggregate variation between said plurality of first response times and said plurality of second response times exceeds said variation threshold.

102. The method of claim 100, wherein determining whether said variation between said first metadata and said second metadata exceeds said variation threshold comprises determining a quantity of queries for which variation between said plurality of first response times and said plurality of second response times exceeds said variation threshold and determining whether said quantity exceeds a quantity threshold.

103. The method of claim 99, wherein (c) comprises determining whether variation between said first plurality of responses and second plurality of responses exceeds a variation threshold.

104. The method of claim 103, wherein determining whether variation between said first plurality of responses and second plurality of responses exceeds said variation threshold comprises determining a quantity of queries for which said first response differs from said second response and determining if said quantity exceeds a quantity threshold.

105. The method of claim 103, wherein determining whether variation between said first plurality of responses and second plurality of responses exceeds said variation threshold comprises determining whether an aggregate variation between said first plurality of responses and said second plurality of responses exceeds said variation threshold.

106. The method of claim 99, wherein (c) comprises, for a query in said plurality of queries, determining whether variation between said first response and said second response exceeds a variation threshold.

107. The method of claim 99, further comprising determining that said reliability is decreased if, for a query in said plurality of queries, said second response time to said query is longer than said first response time to said query.

108. The method of claim 99, further comprising determining that said reliability is increased if, for a query in said plurality of queries, said first response time to said query is equal to or longer than said second response time to said query.

109. The method of claim 99, wherein (a) and (b) comprise administering said survey to a user via a graphical user interface.

110. The method of claim 109, further comprising, between said first session and said second session, prompting said user to perform an activity unrelated to said survey.

111. The method of claim 99, wherein said survey is a mental health or behavioral health survey.

112. The method of claim 99, wherein said first metadata comprises a first order in which said first plurality of responses was generated by a user and said second metadata comprises a second order in which said second plurality of responses was generated by said user.

113. The method of claim 112, wherein said first order is different than said second order.

114. The method of claim 99, wherein said first metadata comprises a first quantity of user corrections to said first plurality of responses and said second metadata comprises a second quantity of user corrections to said second plurality of responses.

115. A system for processing survey responses, comprising:
one or more computer processors; and
memory comprising machine-executable instructions that, upon execution by said one or more computer processors, cause said one or more computer processors to perform a method comprising:
obtaining, during a first session, (i) a first plurality of responses to a plurality of queries in a survey and (ii) first metadata associated with said first plurality of responses, which first metadata comprises a plurality first response times for said first plurality of responses;
obtaining, during a second session, (i) a second plurality of responses to said plurality of queries and (ii) second metadata associated with said second plurality of responses, which second metadata comprises a plurality second response times for said second plurality of responses; and
processing (i) said first plurality of responses and second plurality of responses or (ii) said first metadata and said second metadata, to identify variation, wherein said variation is indicative of a reliability of said first plurality of responses.

116. The system of claim 115, wherein said reliability of said first plurality of responses is determined based on said variation between said first metadata and said second metadata, and wherein (c) further comprises determining whether said variation between said first metadata and said second metadata exceeds a variation threshold.

117. The system of claim 115, wherein determining whether said variation between said first metadata and said second metadata exceeds said variation threshold comprises determining whether an aggregate variation between said plurality of first response times and said plurality of second response times exceeds said variation threshold.

118. A non-transitory computer readable-medium comprising machine-executable instructions that, upon execution by one or more computer processors, cause said one or more computer processors to perform a method comprising:
obtaining, during a first session, (i) a first plurality of responses to a plurality of queries in a survey and (ii) first metadata about said first plurality of responses, which first metadata comprises a plurality first response times for said first plurality of responses;
obtaining, during a second session, (i) a second plurality of responses to said plurality of queries and (ii) second metadata about said second plurality of responses, which second metadata comprises a plurality second response times for said second plurality of responses; and
processing (i) said first plurality of responses and second plurality of responses or (ii) said first metadata and said second metadata, to identify variation, wherein said variation is indicative of a reliability of said first plurality of responses.
